(19) United States
(12) Patent Application Publication
(10) Pub. No.: US 2015/0374581 A1
(43) Pub. Date: Dec. 31, 2015

(54) USE OF AUTOLOGOUS SERUM FOR TRANSPORT OF ISOLATED STROMAL VASCULAR FRACTION OR ADIPOSE DERIVED STEM CELLS FOR REINJECTION

(71) Applicant: Ruth Z. Goldman, Cherry Hill, NJ (US)

(72) Inventor: Ruth Z. Goldman, Cherry Hill, NJ (US)

(21) Appl. No.: 14/250,338

(22) Filed: Apr. 10, 2014

(51) Int. Cl. A61J 1/05; A61K 35/35; A61K 35/12; B65D 81/18

(52) U.S. Cl. CPC A61J 1/05; B65D 81/18; A61K 35/35; A61K 35/12; A61J 2200/40

(57) ABSTRACT

This invention is directed to devices and methods for use of an autologous serum for transport of isolated stromal vascular fraction or adipose derived stem cells for reinjection.

### CROSS REFERENCE TO RELATED APPLICATIONS

This application claims priority under 35 U.S.C. 119(e) to U.S. Provisional Application No. 61/810,970 filed Apr. 11, 2013, incorporated herein in its entirety.

### FIELD OF THE INVENTION

The invention is directed to autologous serum for transport of isolated stromal vascular fraction or adipose derived stem cells for reinjection.

### BACKGROUND OF THE INVENTION

Biological specimens are often collected, transported and stored for later use as therapeutics or cosmetics. Conventionally, liquid suspensions of biological specimens are stored in sealed tubes under refrigeration. Liquid sample collection, handling, transportation and storage has had many associated problems, most particularly cost. More particularly, the costs associated with refrigeration in storage and collection centers and collection of adequate sample volume to ensure quantities compatible with laboratory methods of subsequent analyses. Further, the risk of container breakage or leakage which results in the loss of sample and the danger of infection and sample instability during shipment and storage can incur additional cost issues. Additionally, transport carriers often refuse to accept biohazard shipments based on established regulations.

Thus, there is a need for a safe, convenient and simple device for collection, storage and transportation of of biological specimens for application in settings where collection, centrifugation, storage and shipment are required as a prerequisite for use as therapeutics and cosmetics.

### BRIEF SUMMARY OF THE INVENTION

The basic design for this process is to collect two tubes of blood from the patient (client) either at time of adipose tissue harvest or prior to preparation of either the Stromal Vascular Fraction (SW) or Adipose derived Stem Cells (ASC) for reinjection. The “autologous” serum is extracted from the blood and stored in labeled tubes in a −80″C±5° C. freezer until time of reinjection. When a request is received from a patient, the request is processed for the SW or ASC is removed from the vapor storage tank and thawed using the quick thaw method (as provided herein). The thawed SVF or ASC is washed two times with Lactated Ringer's Solution, removing the supernatant between washes. After the first wash and re-suspension of the subsequent pellet, an aliquot will be removed for a cell count and viability assay. After the second wash, the cells will be reconstituted in the stored autologous serum and transferred to a sterile syringe and capped with a sterile syringe cap. The syringe is then packaged so that the plunger is secured and is shipped chilled to the physician who submitted the request from the patient.

In a first embodiment, the invention is directed to a biomedical transportation system for use by a patient. The biomedical transportation system includes autologous serum, a biomedical component suspended in the autologous serum, a vessel holding the biomedical component suspended in the autologous system and a temperature control device and a shipment container.

In another embodiment, the invention is directed to a process to prepare a biomedical transport system including the steps of preparing an autologous serum, preparation of a biomedical component, suspension of the biomedical component in the autologous serum, preparation of vessel for holding the biomedical component in the autologous serum, and preparation of a climate controlled shipment container.

In yet another embodiment, the invention is directed to a business method for the collection, storage and transportation of a biomedical sample for therapeutic use including the steps of obtaining a biomedical sample from a patient by a physician, delivering the biomedical sample to a process laboratory, digesting the biomedical sample to form a biomedical component; combining the biomedical component with autolgous serum obtained from the patient to form a cells-autologous serum biomedical transport system, transporting the biomedical transport system to a physician, separating the biomedical transport system to obtain the biomedical component, and injecting the biomedical into the patient from whom the biomedical sample was obtained.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The embodiments discussed herein are not intended to be exhaustive or to limit the invention to the precise form disclosed. These embodiments are chosen and described to best explain the principle of the invention and its application and practical use and to enable others skilled in the art to best utilize the invention.

In a first embodiment, the invention is directed to a biomedical transportation system for use by a patient including an autologous serum obtained from a patient's blood. A biomedical component, usually in the form of stem cells or a stomal vascular fraction (SVF) is suspended in the autologous serum. A vessel holding the biomedical component suspended in the autologous system is provided. The vessel is included with a temperature control device, which are both placed in a shipment container.

In another embodiment, the invention is directed to a process to prepare a biomedical transport system including the steps of preparing an autologous serum obtained from a patient's blood. Preparation of a biomedical component which can be of a variety of tissue including stem cells or SVF obtained from adipose tissue. Suspension of the biomedical component in the autologous serum and preparation of a vessel for holding the biomedical component in the autologous serum is established; the biological transportation system. The preparation of a climate controlled shipment container is created for the biological transportation system.

In yet another embodiment, the invention is directed to business method for the collection, storage and transportation of a biomedical sample for therapeutic use including the steps of obtaining a biomedical sample from a patient by a physician, delivering the biomedical sample to a process laboratory, digesting the biomedical sample to form a biomedical component; combining the biomedical component with autolgous serum obtained from the patient to form a cells-autologous serum biomedical transport system, transporting the biomedical transport system to a physician, separating the biomedical transport system to obtain the biomedical component, and injecting the biomedical into the patient from whom the biomedical sample was obtained.

### EXAMPLES

### Autologous Serum Preparation

Obtain two tubes of 8-10 mL each of the patient's blood, using a blood tube containing either no additive or a clot activator; such blood tubes include Bectin Dickinson (BD) Vacutainer Plus plastic serum tube, 10 ml, red top (#367820), or BD Vacutainer Plus plastic serum tube (transport tube), 10 ml, mottled red/grey top (#367985). At time of receipt to the processing laboratory, the blood is centrifuged at 2350 rpm (1300 g) for 15 minutes at a temperature setting of 8° C. with the break off. The supernatant (top layer containing the serum) will be collected in a centrifuge tube; at a total yield volume of about 6-8 mL The supernatant is dispensed in 1-3 mL aliquots in cryogenic vials. The vials, labeled specifically for that patient's use only, will be stored in a −80° C±5° C. freezer. The serum can be stored for at least two years. Preparation of Product's Transport Vessel

### Direct Transport of Cryopreserved SVF or ASC

Remove frozen sample from liquid nitrogen vapor storage and thaw in a 37° C. water bath using the quick thaw method. Transfer cells to a centrifuge tube containing 10 mL of Lactated Ringer's Solution (LR) and centrifuge at 1000 rpm (228 g) for 10 minutes at 10° C. Remove supernatant (may use this for bacterial test sample). Reconstitute cell pellet in 5 mL LR and remove a 20 μL sample, perform a viability cell count using AO-PI dual fluorescence stain to ensure the cells' viability and to determine the actual number of cells available for reinjection. Centrifuge again at 1000 rpm (228 g) for 10 minutes at 10° C. and remove supernatant. Remove patient's previously prepared serum from −80° C±5° C. freezer and thaw in 37° C. water. Re-suspend cell pellet in this serum. Load cells-autologous serum mixture into a sterile syringe and attach a sterile luer lock cap. Wrap for syringe for transport so that the plunger is secure. Ship on cold packs to requesting physician.

### Expansion and Transport of ASC

Remove frozen sample from liquid nitrogen vapor storage and thaw in a 37° C. water bath using the quick thaw method. Transfer cells to a centrifuge tube containing 5 mL HSA-ASC Medium and remove a 20 μL sample to perform a viability cell count using AO-PI dual fluorescence stain to determine the culture flask plating. Centrifuge again at 1000 rpm (228 g) for 10 minutes. Remove supernatant (may use this for bacterial test sample). Plate cells in fibronectin coated culture flasks as determined by the cell count, in HSA-ASC Medium and incubate in a humidified 5% CO₂, 37° C. incubator. Cells will be grown until requested cell yield is achieved. Harvest cell (ASC) from culture flask using D-PBS, Tryple Select-CTS, and Defined Trypsin Inhibitor. Remove a 20 μL sample and perform a viability cell count using AO-PI dual fluorescence stain. Centrifuge at 1000 rpm (228 g) for 10 minutes at 10° C. and remove supernatant (may use this for bacterial test sample). Reconstitute pellet in 10 mL LR as a wash step and centrifuge again as above; remove supernatant. Remove patient's previously prepared serum from −80° C±5° C. freezer and thaw in 37° C. water. Re-suspend cell pellet in patient's previously prepared serum. Load cells-autologous serum mixture into a sterile syringe and attach a sterile luer lock cap. Wrap for syringe for transport so that the plunger is secure. Ship on cold packs to requesting physician.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A biomedical transportation system for use by a patient comprising:
a. autologous serum;
b. biomedical component suspended in the autologous serum;
c. a vessel holding the biomedical component suspended in the autologous system;
d. a temperature control device; and
e. a shipment container.

2. The biomedical transportation system of claim 1, wherein the biomedical component is autologous stem cells or stromal vascular fraction (SVF).

3. The biomedical transportation system of claim 1, wherein the autologous serum is derived from a blood obtained.

4. The biomedical transportation system of claim 1, wherein the autologous stem cells or stromal vascular fraction (SVF) and autologous serum is obtained from the same source.

5. The system of claim 1, wherein the cells are expanded prior to suspension into the autologous serum.

6. A process to prepare a biomedical transport system comprising the steps of :
a. preparing an autologous serum;
b. preparation of a biomedical component;
c. suspension of the biomedical component in the autologous serum;
d. preparation of a vessel for holding the biomedical component in the autologous serum; and
e. preparation of a climate controlled shipment container.

7. A business method for the collection, storage and transportation of a biomedical sample for therapeutic use comprising the steps of:
a. obtaining a biomedical sample from a patient by a physician;
b. delivering the biomedical sample to a process laboratory;
c. digesting the biomedical sample to form a biomedical component;
d. combining the biomedical component with autolgous serum obtained from the patient of step a. to form a cells-autologous serum biomedical transport system;
e. transporting the biomedical transport system to a physician;
f. separating the biomedical transport system to obtain the biomedical component; and
g. injecting the biomedical into the patient from whom the biomedical sample was obtained in step a.

8. The business method of claim 7, wherein biomedical component is cryogenically stored.

9. The business method of claim 8, wherein the autologous serum is cryogenically stored

10. The business method of claim 9, wherein biomedical component is expanded in HSA-ASC medium upon thawing from cryogenic storage.

11. The business method of claim 10, wherein the cells-autologous biomedical transportation system is put into a sterile syringe and a sterile luer lock cap is attached.

12. The business method of claim 11, wherein the sterile syringe is wrapped for transport so that a plunger on the sterile storage is secured and shipped on cold packs to a physician for injection into the patient of step a.
